(19) United States
(12) Patent Application Publication
(10) Pub. No.: US 2024/0358120 A1
(43) Pub. Date: Oct. 31, 2024

(54) METHODS, APPARATUSES, AND SYSTEMS FOR GENERATING INSOLES

(71) Applicant: Groov, Lake Success, NY (US)

(72) Inventor: Daniel Cataldi, Lake Success, NY (US)

(21) Appl. No.: 18/646,361

(22) Filed: Apr. 25, 2024

Related U.S. Application Data
(60) Provisional application No. 63/498,310, filed on Apr. 26, 2023.
(60) Provisional application No. 63/606,366, filed on Dec. 5, 2023.

(51) Int. Cl. A43D 1/02; A61B 5/00; A61B 5/107

(52) U.S. Cl. CPC A43D 1/025; A61B 5/1074; A61B 5/7435

(57) ABSTRACT

Methods, apparatuses, and systems are described for scanning feet of an individual for generating insoles for shoes of the individual. The individual may create a profile for storing information associated with the individual, including shoe information and the scans of the feet of the individual. The insoles may be generated based on the scans of the feet of the individual in addition to the shoe information.

### CROSS REFERENCE TO RELATED PATENT APPLICATIONS

This application claims priority to U.S. Provisional Patent Application No. 63/498,310, filed on Apr. 26, 2023, and to U.S. Provisional Patent Application No. 63/606,366, filed on Dec. 5, 2023, which are hereby incorporated by reference in their entirety.

### BACKGROUND

Shoe insoles, or inserts, are useful for several purposes such as improving daily wear comfort, height enhancement, plantar fasciitis treatment, arch support, foot and joint pain relief from arthritis, preventing overuse, mitigating injuries, assisting in leg length discrepancy, assisting in the recovery from orthopedic correction, and providing assistance in performing athletic activities. Essentially, shoe insoles help treat and prevent foot motion and/or gait problems that affect a person's soles, ankles, knees, hips, back, etc., especially while performing athletic activities where the load on the feet is many times the weight of the individual's body. Shoe insoles designed for athletic use are useful for supporting and stabilizing the foot, as well as for providing additional shock absorption in order to reduce the load on joints. However, ready-made footwear, including footwear insoles, are not customized for individual consumers. In addition, conventional systems and methods for designing and generating customized footwear, including footwear insoles, are generally time consuming, expensive, and antiquated. For example, conventional systems and methods include scanning feet with different types of foot scanners. However, these scanning methods do not take an adequate amount of positional data in order to account for the dynamic nature of the foot. Moreover, these scanning methods do not adequately take into account the nature of how the user intends to wear the insoles.

### SUMMARY

It is to be understood that both the following general description and the following detailed description are exemplary and explanatory only and are not restrictive.

Methods, systems, and apparatuses systems for improved scanning of an individual's feet, footwear, gait, or other footwear-related entities for generating insoles and other personalized footwear-related solutions are described. A data capture device (e.g., smartphone, camera, tablet computer, etc.) connected to a network may generate and/or maintain image scans of an individual's feet for generating insoles. Each foot of an individual may be scanned (e.g., 3-D images) using the data capture device. The scans may be stored in a user profile associated with the individual or a group of individuals. The user profile may also store data associated with shoes of the individual that may be used in addition to the scans of the individual's feet to generate insoles according to a specific shoe of the individual. Insoles may be generated (e.g., produced) based on the scans of the individual's feet. The insoles may also be generated (e.g., produced) based on the data associated with the shoes stored in the individual's user profile in addition to the scans of the individual's feet. In addition, one or more shoe designs may be generated and one or more shoes may be produced based on the generated insoles.

In an embodiment, are methods comprising outputting, by a user device, a first user interface configured to display one or more options for initiating a scanning process of an extremity of a user, causing, based on a user input with the first user interface, one or more scanning devices to be activated and output of a scanning device interface configured to display the extremity of the user as the extremity is scanned according to the scanning process, receiving, from the one or more scanning devices, based on a user input with the scanning device interface, a plurality of scans of the extremity of the user, and outputting, based on the plurality of scans, a second interface configured to display one or more options for producing a pair of insoles.

In an embodiment, are methods comprising receiving, by a device, from one or more scanning devices, a first plurality of scans of an extremity of a user, determining, based on each scan of the first plurality of scans, that data associated with a first one or more scans of the first plurality of scans satisfies a threshold and data associated with a second one or more scans of the first plurality of scans do not satisfy the threshold, causing, based on the data associated with the second one or more scans not satisfying the threshold, the second one or more scans to be retaken until each scan of the second one or more scans satisfies the threshold, determining, based on the first one or more scans and the retaken second one or more scans, a second plurality of scans, and causing, based on the second plurality of scans, a production of a pair of insoles.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of the present description serve to explain the principles of the apparatuses and systems described herein:

FIG. 1 shows an example system;

FIG. 2 shows an example system;

FIG. 3 shows a flowchart of an example scan method;

FIG. 4 shows an example system environment;

FIGS. 5A-5D show example scans of an individual's foot;

FIG. 6 shows an example operational flow of uploading groups of user profiles;

FIG. 7 shows a flowchart of an example method; and

FIG. 8 shows a flowchart of an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

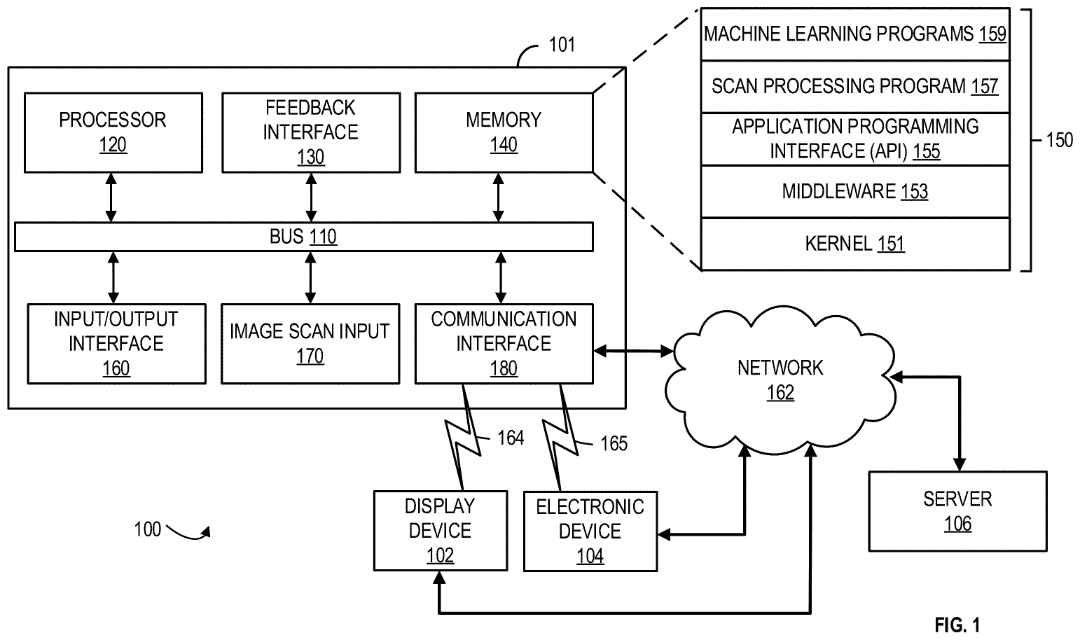

FIG. 1 shows an example system 100 for scanning and generating (e.g., producing) shoe insoles. For example, a device (e.g., data capture device 101) may scan one or more of individual's extremities (e.g., feet, hands, arms, legs, etc.). For example, the device may receive a plurality of scans of one or more of the extremities via one or more scanning devices. The scans may be retaken until each scan satisfies a quality threshold. The scans that satisfy the quality threshold may be used to generate (e.g., produce) a pair of insoles for the individual. The system 100 may include a data capture device 101, a display device 102, an electronic device 104, and one or more servers 106. In an example, the data capture device 101 may be configured to take a plurality of scans of an individual's extremity (e.g., feet, hands, arms, legs, etc.). In an example, the data capture device 101 may be in communication with the display device 102, the electronic device 104, and the one or more servers 106 via a network (e.g., network 162).

The data capture device 101 may include a bus 110, one or more processors 120, a feedback interface 130, a memory 140, an input/output interface 160, an image scan input 170, and a communication interface 180. In certain examples, the data capture device 101 may omit at least one of the aforementioned elements or may additionally include other elements. The data capture device 101 may comprise, for example, a laptop computer, a mobile phone, a smart phone, a tablet computer, a wearable device, a smartwatch, a haptic device, a desktop computer, a smart television, and the like.

The bus 110 may comprise a circuit for connecting the bus 110, the one or more processors 120, the feedback interface 130, the memory 140, the input/output interface 160, the image scan input 170, and/or the communication interface 180 to each other and for delivering communication (e.g., a control message and/or data) between the bus 110, the one or more processors 120, the feedback interface 130, the memory 140, the input/output interface 160, the image scan input 170, and/or the communication interface 180.

The one or more processors 120 may include one or more of a Central Processing Unit (CPU), an Application Processor (AP), or a Communication Processor (CP). The one or more processors 120 may control, for example, at least one of the bus 110, the feedback interface 130, the memory 140, the input/output interface 160, the image scan input 170, and/or the communication interface 180 of the data capture device 101 and/or may execute an arithmetic operation or data processing for communication. As an example, the one or more processors 120 may drive (e.g., cause) the image scan input 170 to take/capture a plurality of scans of an individual's extremity (e.g., feet, hands, arms, legs, etc.). As an example, the one or more processors 120 may drive (e.g., cause) the feedback interface 130 to output feedback (e.g., haptic feedback, audio feedback, visual feedback, etc.) to the individual during the scanning process. For example, the feedback may indicate that the data capture device 101 is continuing to collect data (e.g., scanning data), real-time course-correction indicators as the individual scans the extremity, the scanning process terminated based on an error during the scanning process, and/or the scanning process has completed. The processing (or controlling) operation of the one or more processors 120 according to various embodiments is described in detail with reference to the following drawings.

The processor-executable instructions executed by the one or more processors 120 may be stored and/or maintained by the memory 140. The memory 140 may include a volatile and/or non-volatile memory. The memory 140 may include random-access memory (RAM), flash memory, solid state or inertial disks, or any combination thereof. As an example, the memory 140 may include an Embedded MultiMedia Card (eMMC). The memory 140 may store, for example, a command or data related to at least one of the bus 110, the one or more processors 120, the feedback interface 130, the memory 140, the input/output interface 160, the image scan input 170, and/or the communication interface 180 of the data capture device 101. According to various examples, the memory 140 may store software and/or a program 150 or may comprise firmware. For example, the program 150 may include a kernel 151, a middleware 153, an Application Programming Interface (API) 155, a scan processing program 157, and/or machine learning programs/models 159, and/or the like, configured for controlling one or more functions of the data capture device 101 and/or an external device (e.g., the display device 102 or electronic device 104). At least one part of the kernel 151, middleware 153, or API 155 may be referred to as an Operating System (OS). The memory 140 may include a computer-readable recording medium (e.g., a non-transitory computer-readable medium) having a program recorded therein to perform the methods according to various embodiments by the one or more processors 120. In an example, the memory 140 may store the scans received from the image scan input 170.

The kernel 151 may control or manage, for example, system resources (e.g., the bus 110, the one or more processors 120, the memory 140, etc.) used to execute an operation or function implemented in other programs (e.g., the middleware 153, the API 155, the scan processing program 157, or the machine learning program/model 159). Further, the kernel 151 may provide an interface capable of controlling or managing the system resources by accessing individual elements of the data capture device 101 in the middleware 153, the API 155, the scan processing program 157, or the machine learning program/model 159.

The middleware 153 may perform, for example, a mediation role, so that the API 155, the scan processing program 157, and/or the machine learning programs/models 159 can communicate with the kernel 151 to exchange data. Further, the middleware 153 may handle one or more task requests received from the scan processing program 157 and/or the machine learning programs/models 159 according to a priority. For example, the middleware 153 may assign a priority of using the system resources (e.g., the bus 110, the one or more processors 120, or the memory 140) of the data capture device 101 to at least one of the scan processing program 157 and/or the machine learning programs/models 159. For example, the middleware 153 may process the one or more task requests according to the priority assigned to at least one of the application programs, and thus, may perform scheduling or load balancing on the one or more task requests.

The API 155 may include at least one interface or function (e.g., instruction), for example, for file control, window control, video processing, and/or character control, as an interface capable of controlling a function provided by the scan processing program 157 and/or the machine learning program/model 159 in the kernel 151 or the middleware 153.

As an example, the scan processing program 157 and the machine learning programs/models 159 may be independent of each other or integrally combined, in whole or in part.

The scan processing program 157 may include logic (e.g., hardware, software, firmware, etc.) that may be implemented to process the scans taken by the image scan input 170. The image scan input 170 may comprise an image sensor, a camera, a depth/motion capture sensor (e.g., RGB-D camera), or any device configured to take/capture scans (e.g., three-dimensional scans) of an extremity (e.g., feet, hands, arms, legs, etc.) of an individual. For example, an individual may move the data capture device 101 around the extremity (e.g., foot, hand, arm, leg, etc.) as the image scan input 170 scans the extremity and the data capture device 101 records the data collected by the image scan input 170 (e.g., storing the scans in memory 140). The scanning process may be initiated based on receiving a user input via the input/output interface 160. For example, a user may select an option, via the input/output interface 160, to activate the image scan input 170 and initiate the scanning process. In an example, the scanning process may be initiated automatically based on a positioning of an extremity (e.g., foot, hand, arm, leg, etc.) of an individual in front of the image scan input 170 after an initial activation of the image scan input 170. For example, an individual may select an option, via the input/output interface 160, to initiate the scanning process, and thus, activating the image scan input 170. The individual may place one of the individual's extremities (e.g., foot, hand, arm, leg, etc.) in front of the image scan input 170, wherein the image scan input 170 may automatically initiate the scanning process after detecting that the extremity (e.g., foot, hand, arm, leg, etc.) is in a correct position in front of the image scan input 170. For example, a certain/predetermined area and angle (e.g., position) of the individual's extremity (e.g., foot, hand, arm, leg, etc.) may be required to be detected by the image scan input 170 before the scanning process is initiated. Once the data capture device 101 determines (e.g., detects) that the required area and angle of the individual's extremity (e.g., foot, hand, arm, leg, etc.) is captured by the image scan input 170, the scanning process may automatically begin.

The scans may be collected by both a visible light camera and an infrared depth-mapping system of the image scan input 170. In an example, the scans may be collected based on one or more of an infrared dot blotter, a gyroscope, a light detection and ranging (LiDAR) sensor, etc. As an example, the scans may outside (e.g., lateral arch) and/or inside (e.g., medial arch) portions of the individual's extremity (e.g., foot, hand, arm, leg, etc.). As an example, the image scan input 170 may capture data indicative of one or more positions of the extremity (e.g., foot, hand, arm, leg, etc.) as the individual walks in front of the image scan input 170. For example, the data capture device 101 (e.g., the scan processing program 157) may be configured to include computer vision gait analysis logic that may be implemented to analyze an individual's gait. The data capture device 101 may perform a gait analysis (e.g., supination/pronation assessment) of the individual as the individual walks in front of the image scan input 170 (e.g., towards the image capture input 170 and/or laterally across the image scan input 170) of the data capture device 101. As an example, as the data capture device 101 receives the scans, the data capture device 101 may provide feedback (e.g., haptic feedback, audio feedback, visual feedback, etc.) via the feedback interface 130. The feedback may indicate that the data capture device 101 is continuing to collect data, real-time course-correction indicators as the individual scans the extremity, the scanning process terminated based on an error during the scanning process, and/or the scanning process has completed. In an example, an object mapping program may operate in unison with the machine learning programs/models 159 during the recording process to identify when a threshold amount of data across relevant regions/portions of the individual's extremity (e.g., foot, hand, arm, leg, etc.) has been collected.

The scan processing program 157 may be further configured to determine whether each scan of the completed scans satisfy a quality threshold. For example, the scan processing program 157 may cause the data capture device 101 to process each scan of a plurality of scans received via the image scan input 170 to determine that data associated with a first one or more scans of the first plurality of scans satisfies the quality threshold and that data associated with a second one or more scans of the first plurality of scans do not satisfy the quality threshold.

As an example, the completed scans may be processed via the machine learning programs/models 159 to determine whether each scan satisfies the quality threshold. The machine learning programs/models 159 may include logic (e.g., hardware, software, firmware, etc.) that may be implemented to process the completed scans taken by the image scan input 170. For example, the machine learning programs/models 159 may include logic comprising a plurality of machine learning models. For example, the machine learning programs/models 159 may include one or more of an extremity segmentation model and/or an extremity classification model. The extremity segmentation model may be configured to process the completed scans to determine points within a cloud that make up the extremity, in each scan, and remove the points within the cloud that do not make up the extremity. The extremity segmentation model may then generate a segmented point cloud, for each scan, based on removing the points within the cloud that do not make up the extremity. The segmented point cloud of each scan may then be sent to the extremity classification model, wherein the extremity classification model may determine whether each scan satisfies the quality threshold (e.g., edge clarity threshold, proper positioning threshold, etc.). In an example, the point cloud may be converted to a computer-aided design (CAD) mesh. The mesh may be sent to the extremity classification model, wherein the extremity classification model may determine whether the associated scan satisfies the quality threshold (e.g., edge clarity threshold, proper positioning threshold, etc.). The scans that do not satisfy the quality threshold may then be retaken until the scans satisfy the quality threshold. In an example, the scans that do not satisfy the quality threshold may be stored for further labeling to be used as training data for the machine learning programs/models 159. For example, the scans that do not satisfy the quality threshold may be stored in the memory 140 or may be sent to the sever 106 and stored in one or more databases of the server 106.

The completed scans, including the retaken scans, that satisfy the quality threshold may be stored in a user profile associated with the individual. In an example, the point clouds may be converted to a CAD mesh, wherein the CAD mesh of each scan may be stored in the user profile associated with the individual. For example, the individual may create a user profile for storing the scans to be used for creating/producing insoles for the individual. The user profile may be stored in a database, such as a database of the server 106. In an example, the individual may store specific shoes in the user profile. As an example, the insoles may be created/produced (e.g., customized) for each of the individual's shoes stored in the individual's user profile. In an example, the individual may input additional user information to the user profile. For example, the additional information may comprise one or more of a height, a weight, a desired length of the pair of insoles to be produced, a desired upper material, or an identifier of the pair of insoles to be produced. The additional information may be used to further customize the insoles for the individual. In an example, a self-augmenting fit profile based on a computer vision wear assessment may be implemented by the scan processing program 157 by scanning a pair of insoles of the individual that have been worn by the individual for a period of time (e.g., days, weeks, months, years, etc.). The fit profile may be stored in the individual's profile to be used with the one or more scans of an individual's for further enhancing the design of the individual's insoles. For example, insoles may be produced/created based on the one or more scans and the fit profile of the individual.

In an example, one or more shoe designs may be generated based on the insoles and/or based on the scans of the individual's feet. For example, one or more types of shoe designs (e.g., sneakers, dress shoes, high heel shoes, running shoes, soccer shoes, football shoes, etc.) may be generated based on the insoles and/or based on the scans of the individual's feet. One or more shoes may be produced based on the one or more shoe designs. The shoe designs (e.g., according to one or more types of shoes designs) may be stored in the user profiled of the individuals. The shoes designs may be sent to one or more manufactures or shoe producers, wherein one or more shoes may be produced based on one or more of the shoe designs. The generated shoe designs may enable individual customers to receive consistent and optimized shoe (e.g., customized shoe) across one of more different types of footwear, brands, shoe sizes, and models. In addition, the generated shoe designs may combine on-demand manufacturing of the insole with just-in-time assembly of the shoe based on the insole.

The input/output interface 160 may include an interface for delivering an instruction or data input from the individual (e.g., an operator of the data capture device 101) or from a different external device (e.g., electronic device 104) to the different elements of the data capture device 101. The input/output interface 160 may further include an interface for outputting one or more user interfaces to the individual. For example, the input/output interface 160 may comprise a display, such as a touch screen display, and/or one or more physical input interfaces (e.g., keyboard, mouse, etc.) configured to receive user inputs. The input/output interface 160 may be configured to output (e.g., display) a first user interface comprising one or more options for initiating the scanning process of an extremity (e.g., foot, hand, arm, leg, etc.) of the individual. In an example, the first user interface may be based on the user profile of the individual. For example, the first user interface may include previously completed scans of one or extremities of the individual and/or shoes previously uploaded by the individual. In an example, the first user interface may include an option to view instructional content before initiating the scan. In an example, the one or more options may include an option for an assisted scanning process and/or an unassisted scanning process. For example, based on a selection of at least one of the one or more options (e.g., the assisted scanning process option, the unassisted scanning process option, etc.), the scanning interface may output (e.g., display) instructions to the individual for positioning of the extremity as the extremity is being scanned.

The individual may select an option to activate one or more scanning devices (e.g. image scan input 170) and initiate the scanning process. In an example, the scanning process may be initiated automatically based on a positioning of an extremity (e.g., foot, hand, arm, leg, etc.) of an individual in front of the image scan input 170 after an initial activation of the image scan input 170. For example, the individual may select an option, via the input/output interface 160, to initiate the scanning process, and thus, activating the image scan input 170. The individual may place one of the individual's extremities (e.g., foot, hand, arm, leg, etc.) in front of the image scan input 170, wherein the image scan input 170 may automatically initiate the scanning process after detecting that the extremity (e.g., foot, hand, arm, leg, etc.) is in a correct position in front of the image scan input 170. For example, a certain/predetermined area and angle (e.g., position) of an individual's extremity (e.g., foot, hand, arm, leg, etc.) may be required to be captured by the image scan input 170 before the scanning process is initiated. Once the data capture device 101 determines (e.g., detects) that the required area and angle of the individual's extremity (e.g., foot, hand, arm, leg, etc.) is captured by the image scan input 170, the scanning process may automatically begin. The input/output interface 160 may output (e.g., display) a scanning device interface. The scanning device interface may be configured to output (e.g., display) the extremity (e.g., foot, hand, arm, leg, etc.) of the individual as the extremity is being scanned according to the scanning process or as the extremity is captured for initiating the scanning process. For example, the input/output interface 160 may output (e.g., display) a visual intake (e.g., three-dimensional scan/image) of the extremity as the extremity is being scanned/captured. In an example, the data capture device 101 may include an infrared dot-blotter within a TrueDepth Camera system (e.g., the image scan input 170). The individual may provide input via the scanning device interface (e.g., via a touch screen interface or one or more physical buttons) to execute each scan of the extremity. The data capture device 101 may receive, via the image scan input 170, a plurality scans for each extremity of the individual and record the scans in the memory 140 and/or may send the scans to the server 106 to be stored in one or more databases in the individual's user profile. For example, the image scan input 170 may scan the individual's extremity as the individual moves the data capture device 101 in a specified motion based on specific areas of coverage around the extremity. In an example, the data capture device 101 may provide feedback (e.g., haptic feedback, audio feedback, visual feedback, etc.), via the feedback interface 130 as the individual scans the extremity. For example, the feedback may indicate that the data capture device 101 is continuing to collect data (e.g., scanning data), real-time course-correction indicators as the individual scans the extremity, the scanning process terminated based on an error during the scanning process, and/or the scanning process has completed.

Based on the plurality of scans, the image scan input 170 may output (e.g., display) a second user interface comprising one or options for producing a pair of insoles. For example, the image scan input 170 may output the one or more options after the scanning process is completed. The one or more options for producing the pair of insoles may comprise a general-purpose shape option or a shoe-specific shape option. Based on a selection of the shoe-specific shape option, a third user interface may be output to the individual. As an example, the third interface may be configured to display instructions for the individual to scan a sockliner or an insole designed to fit in one or more shoes. As an example, the third user interface may be configured to receive user input associated with additional information of the user. The additional information may comprise one or more of a height, a weight, a desired length of the pair of insoles to be produced, a desired upper material, or an identifier of the pair of insoles to be produced. In an example, based on the selection of the shoe-specific shape option, the pair of insoles may be produced according to the plurality of scans and the data associated with the one or more shoes associated with the individual.

In an example, an additional user interface may be provided for producing one or more pairs of shoes. The one or more pairs of shoes may be produced based on the generated insole/sockliner and/or based on the scans of the individual's feet. In addition, the one or more pairs of shoes may be generated based on one or more of the height, the weight, the desired length of the pair of insoles to be produced, or the desired upper material. For example, one or more shoe designs may be generated based on the insoles/scans associated with the individual's feet and/or based on one or more of the height, the weight, the desired length of the pair of insoles to be produced, or the desired upper material. The shoe designs (e.g., according to one or more types of shoes designs) may be stored in the user profiled of the individuals. The shoes designs may be sent to one or more manufactures or shoe producers, wherein one or more shoes may be produced based on one or more of the shoe designs.

In an example, the input/output interface 160 may output an instruction or data received from one or more elements of the data capture device 101 to one or more external devices (e.g., display device 102 or electronic device 104).

The communication interface 180 may establish, for example, communication between the data capture device 101 and one or more external devices (e.g., the display device 102, the electronic device 104, and/or the server 106). For example, the communication interface 180 may communicate with the one or more external devices (e.g., the display device 102, the electronic device 104, and/or the server 106) by being connected to a network 162 through wireless communication or wired communication. The network 162 may include, for example, at least one of a telecommunications network, a computer network (e.g., LAN or WAN), the Internet, and/or a telephone network.

The communication interface 180 may be configured to communicate with the one or more external devices (e.g., display device 102, or electronic device 104) via a wired communication interface 164, 165 or a wireless communication interface 164, 165. In an example, the wired communication may include, for example, at least one of Universal Serial Bus (USB), High Definition Multimedia Interface (HDMI), Recommended Standard-232 (RS-232), power-line communication, Plain Old Telephone Service (POTS), and the like. In an example, as a cellular communication protocol, the wireless communication interface 164, 165 may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the wireless communication interface 164, 165 may be configured to use a near-distance communication 164, 165. The near-distance communication interface 164, 165 may include for example, at least one of Wireless Fidelity (WiFi), Bluetooth, Bluetooth Low Energy (BLE), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (GLONASS), BeiDou Navigation Satellite System (BDS), Galileo, the European global satellite-based navigation system, and the like. Hereinafter, the “GPS” and the “GNSS” may be used interchangeably in the present document. In an example, the communication interface 180 may include or be communicably coupled to a transmitter, receiver and/or transceiver for communication with the external devices (e.g., display device 102, or electronic device 104).

The display device 102 may comprise one or more of a smart television, an audio/video monitor, a streaming device, and the like. The display device 102 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. In an example, the display device 102 may be configured as a part of the data capture device 101 or as a separate device. The display device 102 may display, for example, a variety of contents (e.g., text, image, video, icons, symbols, etc.) to the individual. For example, the display device 102 may be configured to output one or more of the first user interface, the scanning device interface, and/or the second user interface output by the input/output interface 160. For example, the data capture device 101 may be configured to send the interfaces to the display device 102 for the display device 102 to output the interfaces to the individual instead of, or in addition to, the data capture device 101.

The electronic device 104 may comprise, for example, a laptop computer, a mobile phone, a smart phone, a tablet computer, a wearable device, a smartwatch, a haptic device, a desktop computer, a smart television, and the like. As an example, the electronic device 104 may be configured to output one or more of the first user interface, the scanning device interface, and/or the second user interface output by the input/output interface 160. For example, the data capture device 101 may be configured to send the interfaces to the electronic device 104 for the electronic device 104 to output to the interfaces to the individual instead of, or in addition to, the data capture device 101.

As an example, the electronic device 104 may comprise an image sensor, a camera device, a smart camera, an infra-red sensor, a depth/motion-capture sensor (e.g., RGB-D camera), a LiDAR sensor, and the like. For example, the electronic device 104 may be configured to capture the scans of the extremities (e.g., feet, hands, arms, legs, etc.), based on input received from the data capture device 101, and send the captured scans to the data capture device 101 for further processing. In an example, the electronic device 104 may be configured to provide the feedback to the individual during the scanning process. In an example, the electronic device 104 may send the completed scans to the data capture device 101, wherein the data capture device 101 may perform the process of determining whether the scans satisfy the quality threshold in order to determine whether any of the scans need to be retaken.

The server 106 may include a group of one or more servers. For example, all or some of the operations executed by the data capture device 101 may be executed in a different one or a plurality of electronic devices (e.g., the display device 102, the electronic device 104, and/or the server 106). In an example, if the data capture device 101 needs to perform a certain function or service either automatically or based on a request, the data capture device 101 may request at least some parts of functions related thereto alternatively or additionally to a different electronic device (e.g., the display device 102, the electronic device 104 and/or the server 106) instead of executing the function or the service autonomously. The different electronic devices (e.g., the display device 102, the electronic device 104, or the server 106) may execute the requested function or additional function, and may deliver a result thereof to the data capture device 101. The data capture device 101 may provide the requested function or service either directly or by additionally processing the received result. For example, a cloud computing, distributed computing, or client-server computing technique may be used.

In an example, the server 106 may include one or more databases. For example, the databases may be used to store a plurality of user profiles associated with a plurality of individuals. Scans associated with each individual may be stored in each individual's user profile. In an example, each individual may store one or more specific shoes (e.g., including shoe sizes and dimensions, shoe brands, shoe types, etc.) in each individual's user profile. As an example, the individual may obtain pairs of shoe insoles that are customized to the individual's feet and/or specific shoes stored in the individual's user profile based on one or more scans of the individual's feet stored in the individual's user profile. In an example, each individual's user profile may further include additional information associated with the individual that may be used for creating/producing the insoles of the individual. For example, the additional information may comprise one or more of a height, a weight, a desired length of the pair of insoles to be produced, a desired upper material, or an identifier of the pair of insoles to be produced. In an example, each individual's user profile may further include information associated with one or more shoe designs based on the generated insoles/scans associated with the individual's feet and/or based on the additional information. In an example, a group (e.g., school, business, organization, etc.) may create a group profile associated with individuals of the group. For example, a school may create a group profile of individuals of different sports teams, organizations, etc. The group may store one or more scans associated with each individual of the group within the group profile in addition to one or more specific shoes (e.g., including shoe sizes and dimensions) associated with the group and/or individuals of the group. As an example, the group may obtain pairs of shoe insoles that are customized for each individual's feet and/or for specific shoes stored in the group's profile associated with each individual based on the one or more scans of each individual's feet stored in the group's profile. In an example, the group's profile may include additional information (e.g., height, weight, desired length of the pair of insoles to be produced, desired upper material, identifier of the pair of insoles to be produced, etc.) associated with each individual of the group that may be used for creating/producing the insoles of the individuals of the group.

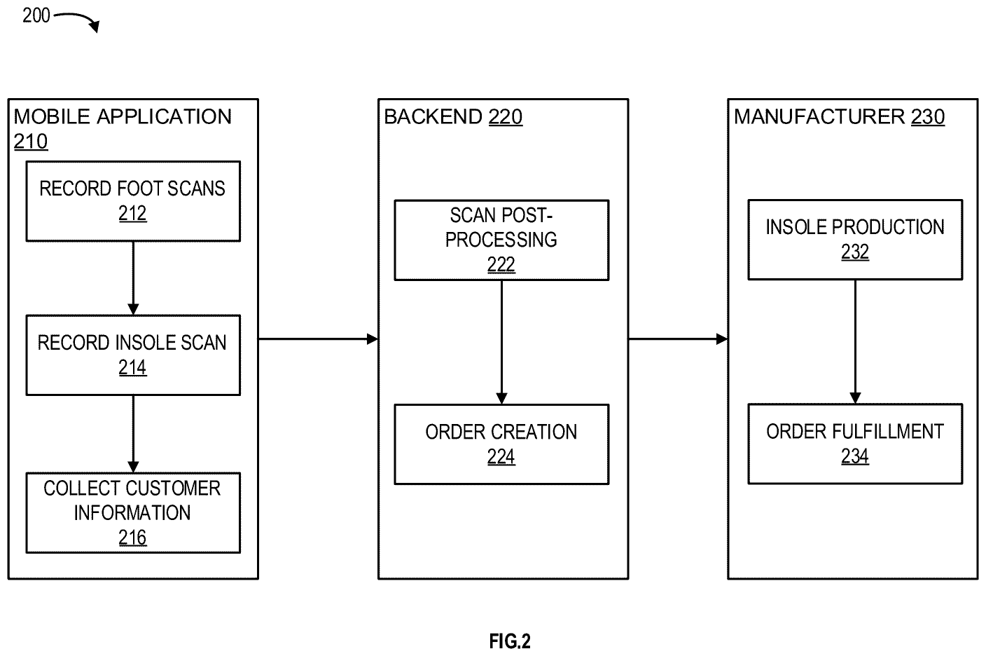

FIG. 2 shows an example system 200 for producing a pair of insoles. The system 200 may comprise a mobile application 210, a backend 220, and a manufacturer 230. The mobile application may be implemented by a user device (e.g., data capture device 101 and/or electronic device 104). As an example, the mobile application may comprise the scan processing program 157 and/or the machine learning programs/models 159. At 212, the mobile application 210 may be configured to process and record scans of an individual's foot. The mobile application 210 may determine whether each scan of the completed scans satisfy a quality threshold. For example, the application may determine that a first one or more scans of the completed scans satisfy the quality threshold and that a second one or more scans of the completed scans do not satisfy the quality threshold. In an example, the mobile application 210 may process the scans via one or more machine learning models (e.g., the machine learning programs/models 159) such as an extremity (e.g., foot) segmentation model and/or an extremity (e.g., foot) classification model. The extremity segmentation model may be configured to process the completed scans to determine points within a cloud that make up the foot, in each scan, and remove the points within the cloud that do not make up the foot. The extremity segmentation model may then generate a segmented point cloud, for each scan, based on removing the points within the cloud that do not make up the foot. The segmented point cloud of each scan may then be sent to the extremity classification model, wherein the extremity classification model may determine whether each scan satisfies the quality threshold (e.g., edge clarity threshold, proper positioning threshold, etc.). The scans that do not satisfy the quality threshold may then be retaken until the scans satisfy the quality threshold. In an example, the scans that do not satisfy the quality threshold may be stored for further labeling to be used as training data for the one or more machine learning programs/models.

In an example, a self-augmenting fit profile based on a computer vision wear assessment may be implemented by scanning a pair of insoles of the individual that have been worn by the individual for a period of time (e.g., days, weeks, months, years, etc.). The fit profile may be stored in the individual's profile to be used with the one or more scans of an individual's for further enhancing the design of the individual's insoles. For example, insoles may be produced/created based on the one or more scans and the fit profile of the individual.

At 214, the mobile application 210 may use the completed scans, including the retaken scans, that satisfy the quality threshold may to generate insole designs/scans for the individual. In an example, the mobile application 210 may also use shoe information (e.g., data) associated with one or more pairs of shoes stored in the individual's user profile to generate the insole designs/scans. For example, the individual may create a user profile for storing the scans for creating/producing insoles for the individual and the shoe information. The insole designs/scans may be stored on the user device or in a database of a server (e.g., server 106) in the individual's user profile. After the insole designs/scans are created, the mobile application 210 may collect customer information of the individual. For example, the mobile application 210 may collect one or more of a height of the individual, a weight of the individual, a desired length of the pair of insoles to be produced, a desired upper material, an identifier of the pair of insoles to be produced, etc.

The mobile application 210 may further send the insole designs/scans to a backend 220 for further processing. As an example, the backend 220 may be implemented by a server (e.g., server 106). The backend 220 may perform insole design/scan post processing at 222.

In an example, part or all of the post processing may be performed by a backend or server associated with the manufacturer 230. In an example, the backend 220 may generate one or more shoe designs based on the generated insole design and/or based on the scans of the individual's feet. For example, one or more types of shoe designs (e.g., sneakers, dress shoes, high heel shoes, running shoes, soccer shoes, football shoes, etc.) may be generated based on the insoles associated with the scans of the individual's feet. Based on the insole design/scan post processing 222, the backend 220 may create orders for one or more pairs of insoles for the individual at 224. In an example, based on the one or more of the generated shoe designs, the backend 220 may create orders for one or more pairs of shoes (e.g., customized shoes) for the individual.

The backend 220 may send the orders of the one or more pairs of insoles and/or the orders for the one or more pairs of shoes to the manufacturer 230. At 232, the manufacturer 230 may produce the one or more pairs of insoles based on the insole designs/scans. In an example, the manufacturer 230 may produce the one or more pairs of shoes based on the shoe designs. At 234, the manufacture 230 may fulfill the order by sending the one or more pairs of insoles and/or the one or more pairs of shoes to the individual. In an example, the mobile application 210 may provide an option to send the pair of insoles, the insole design, and/or the scans to a store (e.g., company, organization, etc.). The store may establish a store-specific user profile, wherein the store may provide custom insoles for the user based on the user profile, such as based on user preferences for certain shoe brands, types, etc. In an example, the backend 220 may be configured to integrate functions with one or more third-party applications (e.g., branded e-commerce applications associated with one or more retail distributors, stores, etc.).

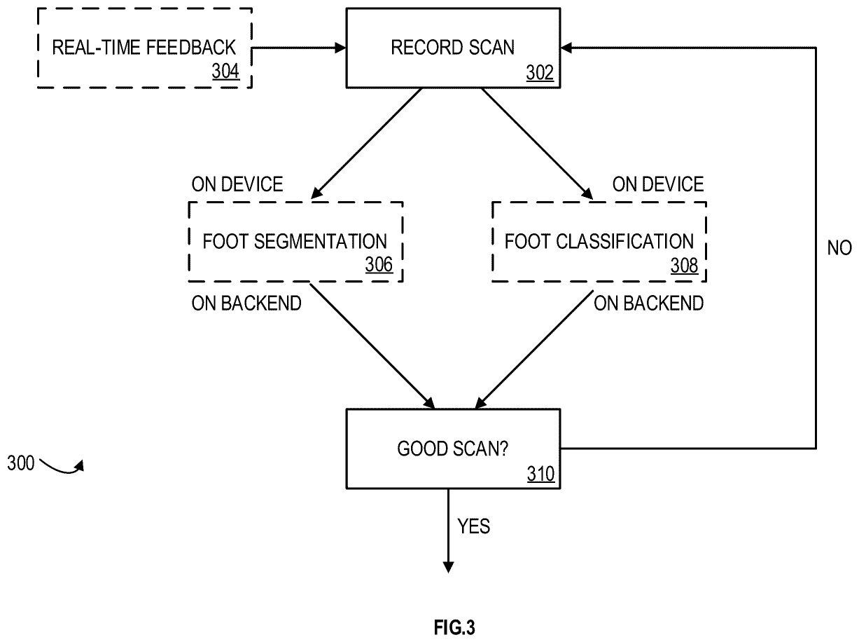

FIG. 3 shows a flowchart of an example scan method 300. At 302, one or more scans of an individual's foot (e.g., left foot or right foot) may be recorded. For example, a user device (e.g., data capture device 101, electronic device 104, etc.) may collect the scans of the individual's foot. In an example, the user device may collect scans of insoles associated with the individual. In an example, feedback may be received during the scanning process of the individual's foot, at 304. The feedback may indicate that the user device is continuing to collect data, real-time course-correction indicators as the individual scans the extremity, the scanning process terminated based on an error during the scanning process, and/or the scanning process has completed. The scans may be processed in order to determine whether each of the scans satisfy a quality threshold. For example, the scans may be processed via one or more machine learning models (e.g., the machine learning programs/models 159) such as a foot (e.g., extremity) segmentation model, at 306, and/or a foot (e.g., extremity) classification model, at 308. For example, at 306, the foot segmentation model may be configured to process the scans to determine points within a cloud that make up the foot (e.g., extremity), in each scan, and remove the points within the cloud that do not make up the foot (e.g., extremity). The foot (e.g., extremity) segmentation model may then generate a segmented point cloud, for each scan, based on removing the points within the cloud that do not make up the extremity. At 308, the segmented point cloud of each scan may be processed by the foot (e.g., extremity) classification model, wherein the foot (e.g., extremity) classification model may determine whether each scan satisfies the quality threshold (e.g., edge clarity threshold, proper positioning threshold, etc.). At 310, the scans that do not satisfy the quality threshold may then be retaken, repeating the scanning process starting at 302, until the scans satisfy the quality threshold. In an example, when scans are retaken feedback may be provided showing the individual how to take a successful scan. The scans that satisfy the quality threshold may then be collected and stored in a user profile of the individual. For example, the scans may then be used to generate/produce the pairs of insoles for the user.

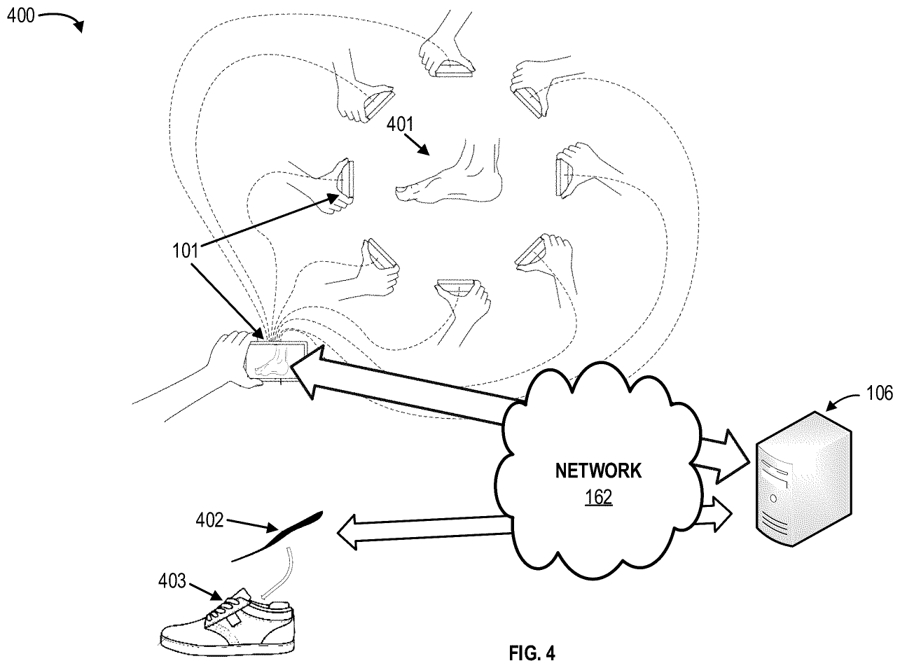

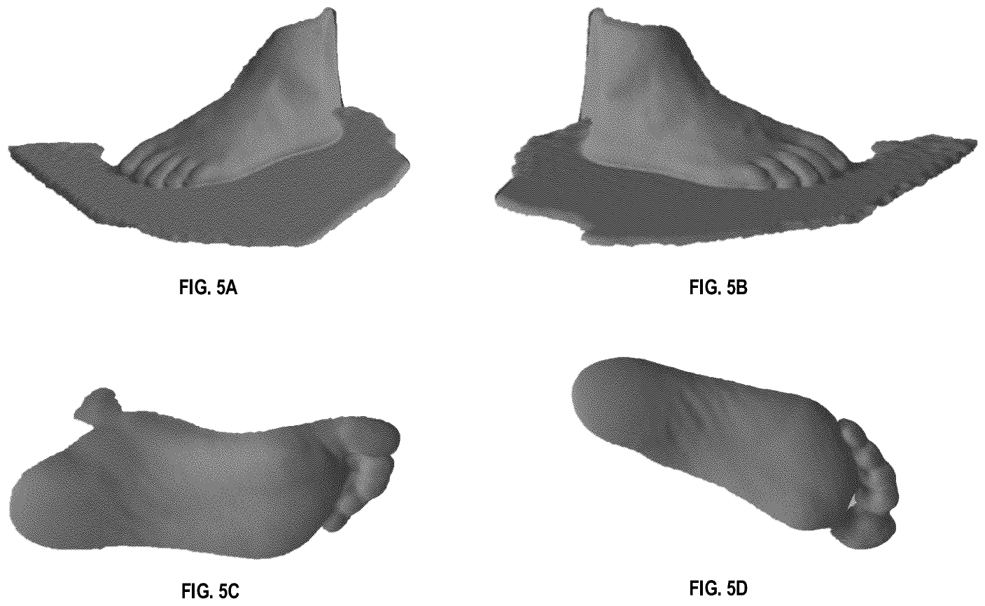

FIG. 4 shows an example system environment 400 for scanning an individual's feet and producing a pair of insoles. A scanning process may be initiated by the data capture device 101. The data capture device 101 may scan the individual's foot (e.g., extremity) 401 as the individual moves the data capture device 101 in a specified motion based on specific areas of coverage around the foot (e.g., extremity) 401, as shown in FIG. 4. In an example, the data capture device 101 may capture data indicative of one or more positions of the extremity (e.g., foot, hand, arm, leg, etc.) as the individual walks in front of the data capture device 101. For example, the data capture device 101 may be configured to analyze an individual's gait. The data capture device 101 may perform a gait analysis of the individual as the individual walks in front of the data capture device 101 (e.g., towards the data capture device 101 and/or laterally across the data capture device 101). As an example, the data capture device 101 may remain stationary while scanning the individual's foot. Feedback (e.g., haptic feedback, audio feedback, visual feedback, etc.) may be received/output during the scanning process of an individual's foot. The feedback may indicate that the data capture device 101 is continuing to collect data, real-time course-correction indicators as the individual scans the extremity, the scanning process terminated based on an error during the scanning process, and/or the scanning process has completed. The completed scans may be stored in a user profile associated with the individual. For example, as shown in FIGS. 5A-5D, the scans may comprise a three-dimensional rendering of the scanned foot or a portion of the scanned foot. FIGS. 5A and 5C show example scans of a left foot and FIGS. 5B and 5D show example scans of a right foot. The three-dimensional image/rendering of the foot may be output (e.g., displayed) to the individual via the data capture device 101. As shown in FIGS. 5A-5D, the three-dimensional image/rendering may be rotated in the display to display the foot from different angles based on user interaction with the three-dimensional image/rendering via the data capture device 101. As an example, the data capture device 101 may send the completed scans to server 106, via network 162, for further processing and to produce pairs of insoles 402 based on the scans. In an example, the scans may be used to create shoe-specific insoles 402 that may be designed/produced according to one or more shoes 403 stored in the user profile of the individual. In an example, the scans and/or the insoles may be used to create one or more pairs of shoes that may be designed based on the scans and/or based on the insoles. In an example, the server 106, may create an order for a pair of insoles based on the scans and send the order to a manufacturer, wherein the manufacturer may produce the insoles and send the insoles to the individual. In an example, the server 106 may create an order for one or more pairs of shoes based on the scans and/or based on the generated insoles and send the order to a manufacturer, wherein the manufacturer may produce the one or more pairs of shoes and send the one or more pairs of shoes to the individual.

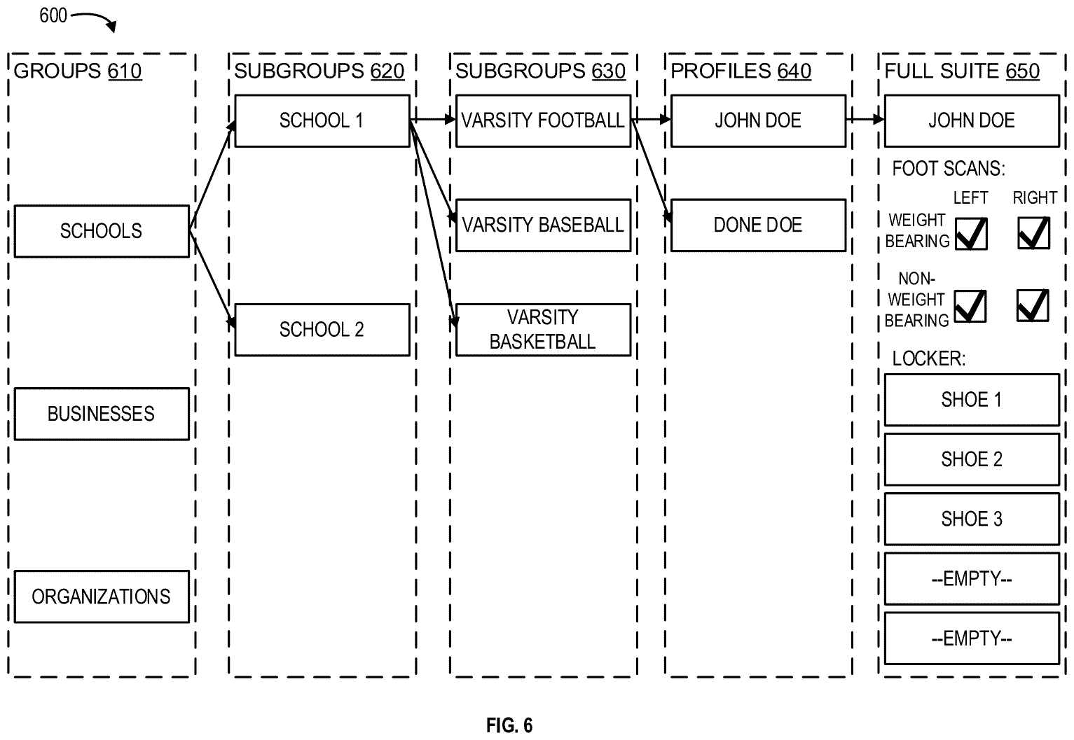

FIG. 6 shows an example operational flow of uploading user profiles that may be associated with different groups or organizations. In an example, one or more groups (e.g., schools, businesses, organizations, associations, etc.) may upload user information associated with one or more individuals of the one or more groups. For example, a group may select that it is associated with a school from a list/database of groups at 610. At 620, a specific school (e.g., subgroup) may be selected. In an example, an individual or group may initiate the application, wherein the application may begin at one of the subgroup 620, 630 selection steps. At 630, based the school, the group may choose the particular subgroup (e.g., sport) associated with the user profiles that the school intends to upload. For example, the school may indicate that the user profiles are associated with the school's varsity football sports team. At 640, the school may indicate, or select, the names of the individuals associated with the user profiles of the varsity football sports team. At 650, the school may update, or create, the user profile information associated with the selected individual. For example, the user profile information may comprise the saved foot scans and a “locker” comprising the shoes uploaded to the selected individual's user profile. The insoles may be produced based on the user profile information. As an example, the group may send/upload the group profile information, comprising user profiles associated with one or more individuals, as a single data file. The data file may be stored on a backend device, such as a server or cloud computing device.

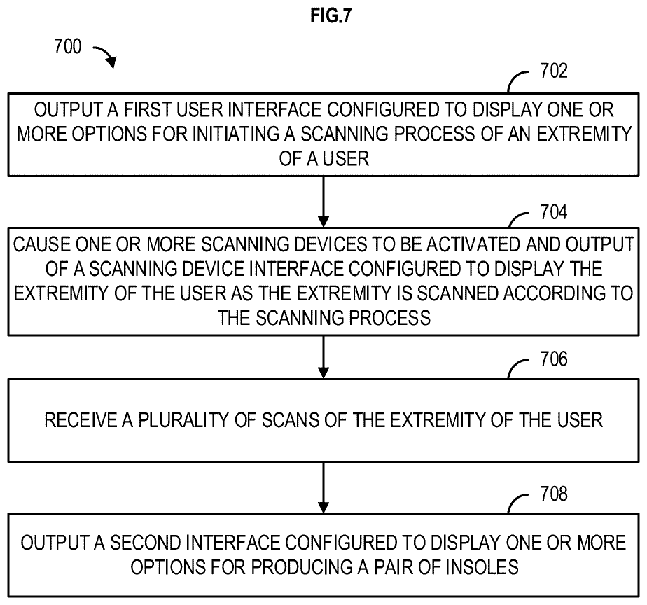

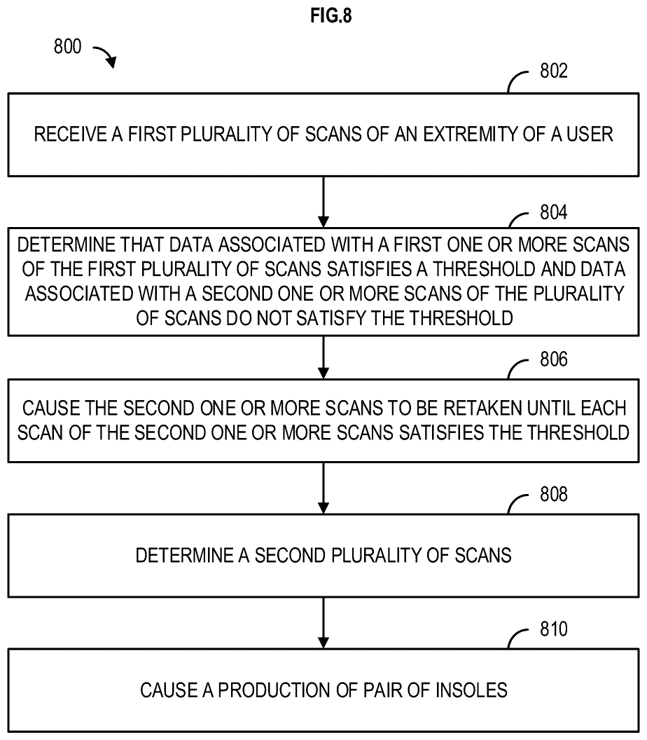

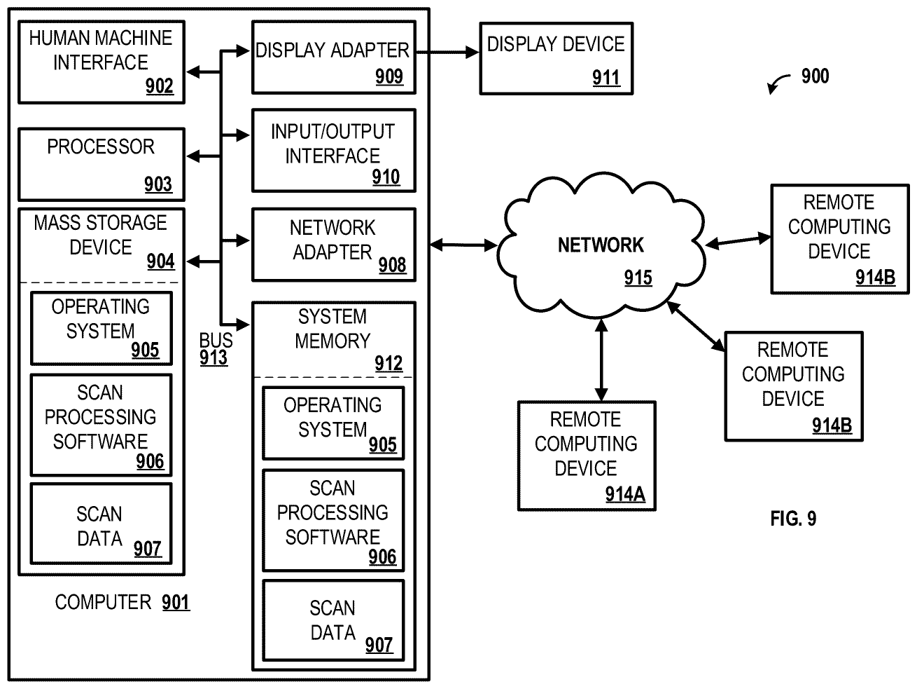

FIG. 7 shows a flowchart of an example method 700 for implementing one or more user interfaces for receiving user input and producing insoles based on one or more scans. Method 700 may be implemented by a user device (e.g., data capture device 101, the electronic device 104, etc.). At step 702, a first user interface may be output (e.g., display) by the user device. The first user interface may comprise one or more options for initiating a scanning process of an extremity of a user. The one or more options for initiating the scanning process may comprise an assisted scanning process or an unassisted scanning process. The extremity may comprise one or more of a left foot of the user or a right foot of the user. In an example, the extremity may comprise a user's arm, hand, leg, limb, etc.

At step 704, one or more scanning devices may be activated and a scanning device interface may be output based on a user input with the first user interface. For example, the one or more scanning devices may be activated and the scanning device interface may be output by the user device (e.g., data capture device 101, the electronic device 104, etc.) based on the user input with the first user interface. The one or more scanning devices may comprise one or more of an imaging device, a camera, a depth camera, or a LiDAR sensor. In an example, the one or more scanning devices may be configured to capture data (e.g., scans) indicative of one or more positions of the extremity as the user walks in front of the one or more scanning devices. For example, the user device may be configured to analyze an individual's gait. The user device may perform a gait analysis of the individual as the individual walks in front of the user device (e.g., towards the user device and/or laterally across the user device). The one or more positions may comprise one or more of a weight-bearing position or a non-weight-bearing position. As an example, the scanning device interface may be further configured to display instructions to the user for positioning of the extremity according to the scanning process based on a selection of at least one option of the one or more options via the first user interface.

As an example, the scanning process may be initiated automatically based on a positioning of an extremity (e.g., foot, hand, arm, leg, etc.) of an individual in front of the user device after an initial activation of the user device. For example, an individual may select an option, via a user interface of the user device, to initiate the scanning process, and thus, activating the user device (e.g., activing the scanning device). The individual may place one of the individual's extremities (e.g., foot, hand, arm, leg, etc.) in front of the user device, wherein the user device may automatically initiate the scanning process after detecting that the extremity (e.g., foot, hand, arm, leg, etc.) is in a correct position in front of the user device. For example, a certain/predetermined area and angle (e.g., position) of an individual's extremity (e.g., foot, hand, arm, leg, etc.) may be required to be detected by the user device before the scanning process is initiated. Once the user device determines (e.g., detects) that the required area and angle of the individual's extremity (e.g., foot, hand, arm, leg, etc.) is captured by the user device, the scanning process may automatically begin.

At step 706, a plurality of scans of the extremity of the user may be received based on a user input with the scanning device interface. For example, the plurality of scans of the extremity of the user may be received by the user device (e.g., data capture device 101, the electronic device 104, etc.) based on the user input with the scanning device interface. In an example, the one or more scanning devices may be configured to output feedback associated with each scan of the plurality of scans. The feedback may comprise one or more of haptic feedback, audio feedback, visual feedback, and the like. For example, the feedback may be indicative of one or more of: the user device is continuing to collect data, real-time course-correction indicators as the individual scans the extremity, the scanning process terminated based on an error during the scanning process, and/or the scanning process has completed. In an example, the feedback may be determined based on an application of an object detection model and a machine learning segmentation model to the plurality of scans.

At step 708, a second user interface may be output based on the plurality of scans. For example, the second user interface may be output by the user device (e.g., data capture device 101, the electronic device 104, etc.) based on the plurality of scans. The second user interface may be configured to display one or more options for producing a pair of insoles. The one or more options for producing the pair of insoles may comprise a general-purpose shape option or a shoe-specific shape option. In an example, a third user interface configured to display instructions for the user to scan a sockliner may be output based on a selection of the shoe-specific shape option. In an example, the third user interface may be configured to receive user input associated with additional information of the user. For example, the additional information may comprise one or more of a height, a weight, a desired length of the pair of insoles to be produced, a desired upper material, or an identifier of the pair of insoles to be produced. In an example, a pair of insoles may be produced based on a selection of the shoe-specific shape option according to the plurality of scans and the data associated with one or more shoes associated with the user. For example, a user may create a user profile associated with the user. The user profile may be stored in a database of user profiles on a server, for example. The scans of the user's extremities may be stored in the user profile for producing the insoles. In addition, the user may upload data associated with one or more pairs of shoes to the user profile. Thus, the insoles may be produced based on the scans of the user's extremities in addition to the data associated with the shoes uploaded to the user profile. In an example, a plurality of user profiles may be stored on a server. The user device may access the server to determine a profile previously created by the user. As an example, the first user interface may be output based on the user profile. For example, the first user interface may be configured to output options associated with information of the user, a “locker” of shoes uploaded by the user, one or more scans previously uploaded by the user, etc. As an example, a fourth interface may be output as the insoles are being produced. For example, the fourth interface may comprise an order-flow management portal that may output updates of the production process of the insoles.

FIG. 8 shows a flowchart of an example method 800 for producing a pair of insoles based on a plurality of scans of a user's extremity. Method 800 may be implemented by a user device (e.g., data capture device 101, the electronic device 104, server 106, etc.). At step 802, a first plurality of scans of an extremity of a user may be received from one or more scanning devices. For example, the plurality of scans of the extremity of the user may be received by the user device (e.g., data capture device 101, the electronic device 104, server 106, etc.) from one or more scanning devices. In an example, a user profile of a plurality of user profiles may be determined. For example, each user profile of the plurality of user profiles may comprise data associated with one or more shoes associated with an individual user. Each user profile may include one or more insoles generated based on a plurality of scans of one or more extremities of a user and the data associated with the one or more shoes associated with the user. The extremity may comprise one or more of a left foot of the user or a right foot of the user. In an example, the extremity may comprise a user's arm, hand, leg, limb, etc. The one or more scanning devices may comprise one or more of an imaging device, a camera, a depth camera, or a LiDAR sensor. In an example, the one or more scanning devices may be configured to capture data indicative of one or more positions of the extremity as the user walks in front of the one or more scanning devices. For example, the user device may be configured to analyze an individual's gait. The user device may perform a gait analysis of the individual as the individual walks in front of the user device (e.g., towards the user device and/or laterally across the user device). For example, the one or more positions may comprise one or more of a weight-bearing position or a non-weight-bearing position. In an example, feedback associated with each scan of the first plurality of scans may be determined. The feedback may comprise one or more of haptic feedback, audio feedback, visual feedback, and the like. The feedback may be indicative of one or more of: the user device is continuing to collect data, real-time course-correction indicators as the individual scans the extremity, a scanning process terminated based on an error during a scanning process, or a scanning process has completed. In an example, the feedback may be determined based on an application of an object detection model and a machine learning segmentation model to the first plurality of scans.

At step 804, it may be determined that data associated with a first one or more scans of the first plurality of scans satisfies a threshold and data associated with a second one or more scans of the first plurality of scans do not satisfy the threshold based on each scan of the first plurality of scans. For example, the user device (e.g., data capture device 101, the electronic device 104, server 106, etc.) may determine that data associated with the first one or more scans of the first plurality of scans satisfies the threshold and data associated with the second one or more scans of the first plurality of scans do not satisfy the threshold based on each scan of the first plurality of scans.

As an example, a first machine learning model and a second machine learning model may be applied to each scan of the first plurality of scans to determine that the data associated with the first one or more scans satisfies the threshold and the data associated with the second one or more scans does not satisfy the threshold. The first machine learning model may comprise an extremity segmentation model and the second machine learning model may comprise an extremity classification model. The first machine learning model may be applied to the first plurality of scans to determine points within a cloud that make up the extremity and remove points within the cloud that do not make up the extremity. Based on removing the points within the cloud that do not make up the extremity, a segmented point cloud may be generated. The second machine learning model may be applied to the segmented point cloud to determine that the data associated with the first one or more scans satisfies the threshold and the data associated with the second one or more scans does not satisfy the threshold.

At step 806, the second one or more scans may be retaken until each scan of the second one or more scans satisfies the threshold based on the data associated with the second one or more scans not satisfying the threshold. For example, the second one or more scans may be retaken by the one or more scanning devices and received by the user device (e.g., data capture device 101, the electronic device 104, server 106, etc.). In an example, the first one or more scans, the second one or more scans, and the retaken second one or more scans may be sent to a computing device. For example, the computing device may comprise a server (e.g., server 106). The first one or more scans, the second one or more scans, and the retaken second one or more scans may be stored in a database of the computing device as labeled training data for training the first machine learning model and the second machine learning model. In an example, the first one or more scans and the retaken second one or more scans may be stored in the databased of the computing device in a user profile associated with the user.

At step 808, a second plurality of scans may be determined based on the first one or more scans and the retaken second one or more scans. For example, the second plurality of scans may be determined by the user device (e.g., data capture device 101, the electronic device 104, server 106, etc.) based on the first one or more scans and the retaken second one or more scans. For example, the second plurality of scans may comprise the first one or more scans and the retaken second one or more scans.

At step 810, a pair of insoles may be produced based on the second plurality of scans. For example, the second plurality of scans may be sent by the user device (e.g., data capture device 101, the electronic device 104, etc.) to the computing device to be used to produce the pair of insoles. The computing device may process the scans in order to create an order for a pair of insoles. The computing device may send the order to a manufacturer, wherein the manufacturer may produce the pair of insoles and send the insoles to the user. In an example, the pair of insoles may be produced based on the second plurality of scans in addition to data associated with one or more pairs of shoes uploaded to a user profile of the user. For example, the user may create a user profile for storing completed scans and data associated with one or more pairs of shoes. In addition, the user may store additional user information in the user profile. For example, the additional user information may comprise one or more of a height, a weight, a desired length of the pair of insoles to be produced, a desired upper material, or an identifier of the pair of insoles to be produced.

The methods and systems can be implemented on a computer 901 as illustrated in FIG. 9 and described below. By way of example, the data capture device 101, the display device 102, the electronic device 104 and/or the server 106 of FIG. 1 and/or the can be a computer 901 as illustrated in FIG. 9. Similarly, the methods and systems disclosed can utilize one or more computers to perform one or more functions in one or more locations. FIG. 9 is a block diagram illustrating an example operating environment 900 for performing the disclosed methods. This example operating environment 900 is only an example of an operating environment and is not intended to suggest any limitation as to the scope of use or functionality of operating environment architecture. Neither should the operating environment 900 be interpreted as having any dependency or requirement relating to any one or combination of components illustrated in the example operating environment 900.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media such as memory storage devices.

Further, one skilled in the art will appreciate that the systems and methods disclosed herein can be implemented via a general-purpose computing device in the form of a computer 901. The computer 901 can comprise one or more components, such as one or more processors 903, a system memory 912, and a bus 913 that couples various components of the computer 901 comprising the one or more processors 903 to the system memory 912. The system can utilize parallel computing.

The bus 913 can comprise one or more of several possible types of bus structures, such as a memory bus, memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 913, and all buses specified in this description can also be implemented over a wired or wireless network connection and one or more of the components of the computer 901, such as the one or more processors 903, a mass storage device 904, an operating system 905, scan processing software 906, scan data 907, a network adapter 908, the system memory 912, an Input/Output Interface 910, a display adapter 909, a display device 911, and a human machine interface 902, can be contained within one or more remote computing devices 914A-914C at physically separate locations, connected through buses of this form, in effect implementing a fully distributed system.

The computer 901 typically comprises a variety of computer readable media. Examples of readable media can be any available media that is accessible by the computer 901 and comprises, for example and not meant to be limiting, both volatile and non-volatile media, removable and non-removable media. The system memory 912 can comprise computer readable media in the form of volatile memory, such as random access memory (RAM), and/or non-volatile memory, such as read only memory (ROM). The system memory 912 typically can comprise data such as the scan data 907 and/or program modules such as the operating system 905 and the scan processing software 906 that are accessible to and/or are operated on by the one or more processors 903.

In another aspect, the computer 901 can also comprise other removable/non-removable, volatile/non-volatile computer storage media. The mass storage device 904 can provide non-volatile storage of computer code, computer readable instructions, data structures, program modules, and other data for the computer 901. For example, the mass storage device 904 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 904, such as, by way of example, the operating system 905 and the scan processing software 906. One or more of the operating system 905 and the scan processing software 906 (or some combination thereof) can comprise elements of the programming and the scan processing software 906. The scan data 907 can also be stored on the mass storage device 904. The scan data 907 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple locations within the network 915.

In another aspect, the user can enter commands and information into the computer 1001 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices can be connected to the one or more processors 903 via the human machine interface 902 that is coupled to the bus 913, but can be connected by other interface and bus structures, such as a parallel port, game port, an IEEE 1394 Port (also known as a Firewire port), a serial port, a network adapter 908, and/or a universal serial bus (USB).

In yet another aspect, the display device 911 can also be connected to the bus 913 via an interface, such as the display adapter 909. It is contemplated that the computer 801 can have more than one display adapter 909 and the computer 901 can have more than one display device 911. For example, the display device 911 can be a monitor, an LCD (Liquid Crystal Display), light emitting diode (LED) display, television, smart lens, smart glass, and/or a projector. In addition to the display device 911, other output peripheral devices can comprise components such as speakers (not shown) and a printer (not shown) which can be connected to the computer 901 via an Input/Output Interface 910. Any step and/or result of the methods can be output in any form to an output device. Such output can be any form of visual representation, comprising, but not limited to, textual, graphical, animation, audio, tactile, and the like. The display device 911 and the computer 901 can be part of one device, or separate devices.

The computer 901 can operate in a networked environment using logical connections to one or more remote computing devices 914A-914C. By way of example, a remote computing device 914A-914C can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 901 and a remote computing device 914A-914C can be made via a network 915, such as a local area network (LAN) and/or a general wide area network (WAN). Such network connections can be through the network adapter 908. The network adapter 908 can be implemented in both wired and wireless environments. Such networking environments are conventional and commonplace in dwellings, offices, enterprise-wide computer networks, intranets, and the Internet.

For purposes of illustration, application programs and other executable program components such as the operating system 905 are illustrated herein as discrete blocks, although it is recognized that such programs and components can reside at various times in different storage components of the computing device 901, and are executed by the one or more processors 903 of the computing device 901. An implementation of the scan processing software 906 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Example computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques comprise, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, such as: matters of logic with respect to arrangement of steps or operational flow; plain meaning derived from grammatical organization or punctuation; the number or type of embodiments described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as examples only, with a true scope and spirit being indicated by the following claims.

What is claimed is:

1. A method comprising:
outputting, by a user device, a first user interface configured to display one or more options for initiating a scanning process of a foot of a user;
causing, based on a user input with the first user interface, one or more scanning devices to be activated and output of a scanning device interface configured to display the foot of the user as the foot is scanned according to the scanning process;
receiving, from the one or more scanning devices, based on a user input with the scanning device interface, a plurality of scans of the foot of the user; and
outputting, based on the plurality of scans, a second user interface configured to display one or more options for producing a pair of insoles.

2. The method of claim 1, wherein the one or more options for initiating the scanning process comprise an assisted scanning process or an unassisted scanning process, and wherein the one or more options for producing the pair of insoles comprise a general-purpose shape option or a shoe-specific shape option.

3. The method of claim 1, further comprising causing, based on a selection of at least one option of the one or more options, the scanning device interface to further display instructions to the user for positioning of the foot based on the scanning process.

4. The method of claim 1, wherein the one or more scanning devices comprise one or more of an imaging device, a camera, a depth camera, or a LiDAR sensor.

5. The method of claim 1, wherein the one or more scanning devices are configured to output feedback associated with each scan of the plurality of scans.

6. The method of claim 5, wherein the feedback comprises one or more of haptic feedback, audio feedback, or visual feedback, and wherein the feedback is indicative of one or more of: the user device is continuing to collect data; real-time course-correction indicators as the individual scans the foot; the scanning process terminated based on an error during the scanning process; or the scanning process has completed.

7. The method of claim 5, wherein the feedback is determined based on an application of an object detection model and a machine learning segmentation model to the plurality of scans.

8. The method of claim 1, further comprising outputting, based on a selection of the one or more options, a third user interface configured to receive user input associated with additional information of the user, wherein the additional information comprises one or more of a height, a weight, a desired length of the pair of insoles to be produced, a desired upper material, or an identifier of the pair of insoles to be produced.

9. The method of claim 1, further comprising:
determining a user profile of a plurality of user profiles, wherein each user profile of the plurality of user profiles comprises data associated with one or more shoes associated with each user; and
outputting the first user interface based on the user profile.

10. The method of claim 9, further comprising outputting the plurality of scans of the foot of the user to a database of the plurality of user profiles, and storing the plurality of scans in the database associated with the user profile.

11. A method comprising:
receiving, by a device, from one or more scanning devices, a first plurality of scans of a foot of a user;
determining, based on each scan of the first plurality of scans, that data associated with a first one or more scans of the first plurality of scans satisfies a threshold and data associated with a second one or more scans of the first plurality of scans do not satisfy the threshold;
causing, based on the data associated with the second one or more scans not satisfying the threshold, the second one or more scans to be retaken until each scan of the second one or more scans satisfies the threshold;
determining, based on the first one or more scans and the retaken second one or more scans, a second plurality of scans; and
causing, based on the second plurality of scans, a production of a pair of insoles.

12. The method of claim 11, further comprising determining a user profile of a plurality of user profiles, wherein each user profile of the plurality of user profiles comprises data associated with one or more shoes associated with a user, and wherein each user profile is associated with insoles generated based on a plurality of scans of one or more extremities of a user and the data associated with the one or more shoes associated with the user.

13. The method of claim 11, wherein the one or more scanning devices comprise one or more of an imaging device, a camera, a depth camera, or a LiDAR sensor.

14. The method of claim 11, wherein determining, based each scan of the first plurality of scans, that the data associated with the first one or more scans satisfies the threshold and the data associated with the second one or more scans do not satisfy the threshold comprises determining, based on an application of a first machine learning model and a second machine learning model to each scan of the first plurality of scans, that the data associated with the first one or more scans satisfies the threshold and the data associated with the second one or more scans do not satisfy the threshold.

15. The method of claim 14, wherein the first machine learning model comprises a foot segmentation model, and wherein the second machine learning model comprises a foot classification model.

16. The method of claim 14, wherein determining, based on the application of the first machine learning model and the second machine learning model to each scan of the first plurality of scans, that the data associated with the first one or more scans satisfies the threshold and the data associated with the second one or more scans do not satisfy the threshold comprises:
determining, based on the application of the first machine learning model to the first plurality of scans, points within a cloud that make up the foot and removing points within the cloud that do not make up the foot;
generating, based on removing the points within the cloud that do not make up the foot, a segmented point cloud; and
determining, based on an application of the second machine learning model to the segmented point cloud, that the data associated with the first one or more scans satisfies the threshold and the data associated with the second one or more scans do not satisfy the threshold.

17. The method of claim 14, further comprising outputting the first one or more scans, the second one or more scans, and the retaken second one or more scans to a computing device, wherein the first one or more scans, the second one or more scans, and the retaken second one or more scans are stored in a database of the computing device as labeled training data for training the first machine learning model and the second machine learning model.

18. The method of claim 11, wherein the first one or more scans and the retaken second one or more scans are stored in a database in a user profile associated with the user.

19. The method of claim 11, further comprising determining, based on an application of an object detection model and a machine learning segmentation model to the first plurality of scans, feedback associated with each scan of the first plurality of scans.

20. The method of claim 19, wherein the feedback is indicative of one or more of: the device is continuing to collect data; real-time course-correction indicators as the individual scans the foot; a scanning process terminated based on an error during a scanning process; or a scanning process has completed.
